(19) United States
(12) Patent Application Publication
(10) Pub. No.: US 2003/0007966 A1
(43) Pub. Date: Jan. 9, 2003

(54) Pharmaceutical preparation for the inhalation of antithrombin in inflammatory lung diseases and ARDS

(76) Inventors: Johannes Hoffmann, Munchen (DE); Christian Wiedermann, Innsbruck (AT); Juergen Roemisch, Marburg (DE)

Correspondence Address: Finnegan, Henderson, Farabow,, Garrett & Dunner, L.L.P., 1300 I Street, N.W., Washington, DC, 20005-3315, US

(21) Appl. No.: 10/188,957

(22) Filed: Jul. 5, 2002

(30) Foreign Application Priority Data
Jul. 6, 2001 (DE) 101 32 307.7

(51) Int. Cl. A61K 38/48; A61K 31/56

(52) U.S. Cl. 424/94.63; 514/171

(57) ABSTRACT

Pharmaceutical preparations for inhalation in inflammatory pulmonary diseases and acute pulmonary failure (=ARDS) are described, by means of which antithrombin III is applied topically.

The invention relates to a pharmaceutical preparation for the prophylaxis and therapy of inflammatory lung diseases and ARDS.

ARDS (=adult respiratory distress syndrome) is a disorder which is also known as shock lung, acute pulmonary failure or acute respiratory insufficiency due to diffuse damage to the alveocapillary membrane. Despite promising new therapeutic strategies, it is still characterized by a very high mortality. In ARDS, a sudden and progressive worsening of the lung function occurs, which is characterized by an increased permeability of the lungs, associated with hypoxemia together with reduced pulmonary compliance and diffuse, bilateral pulmonary infiltrates in the thoracic X-ray photograph. ARDS can occur as a consequence of direct damage to the lung parenchyma or indirectly in association with a number of acute systemic processes (1). The destruction of the pulmonary permeability represents an important part of the acute lung damage, both the chemical composition and the functional activity of the pulmonary surfactant being modified in patients with ARDS (2). These symptoms also occur in similar form in other inflammatory lung diseases.

In the German patent application 44 34 629, compositions for the treatment of IRDS and ARDS have already been specified which contain at least one glucocorticoid and one pulmonary surfactant. The treatment period and the mortality caused by these syndromes can be reduced by pharmaceuticals of this type. From international patent application WO 89/01341, processes and pharmaceuticals for the treatment of lung membrane diseases such as ARDS are already known in which therapeutically active amounts of (-interferon and/or tumor necrosis factor (TNF) are employed either on their own or in combination with corticosteroids. By this means, the formation of pulmonary surfactants is stimulated. The use of antibodies against TNF-V (anti-TNF) for pharmaceuticals for the treatment or prophylaxis of ARDS is known from international patent application WO 91/04054.

Although, owing to the development of new therapeutic strategies of this type, advances have been made in the treatment of inflammatory lung diseases and of ARDS, ARDS is still associated with a high mortality, the therapies up to now largely remaining only supportive. As a causal reason, at present only the possibility of treatment with antibiotics for the sanitization of the source of sepsis offers itself without thereby, however, being able to exert a direct influence on the inflammatory process. A possible explanation of the progressive development of a multiorgan dysfunction following an acute pulmonary failure could be that due to mechanical ventilation an increase in the inflammatory response in the lung occurs and that inflammatory mediator cascades are activated thereby which increase the tissue damage (3). Thus, depending on the degree of inflammation and the phase of ARDS, high local concentrations of mediators (interleukin-6, interleukin-1, tumor necrosis factor (TNF-V)) can be detected in ARDS. The locally released inflammatory cytokines appear here to have a key role in the increase in lung damage.

In large sepsis studies in the last 10 years, specific and nonspecific inflammatory substances such as antibodies against endotoxins and against cytokines (anti-TNF, anti-interleukin-6, interleukin-1RA) and modulators of coagulation (antithrombin III, protein C) have been tested. Using this systemic therapy, however, no decisive improvement in the organ failure parameters or the overall mortality was achieved, high concentrations of the antiinflammatory substances having to be administered systemically in order to achieve therapeutic levels in the target organs, for example the lung. Thus, only under long-term systemic therapy with antithrombin did an improvement occur in the pulmonary function in patients with severe sepsis, which was characterized by the Horowitz coefficient (pa O₂/FIO₂). In this connection, very high daily doses of antithrombin had to be administered systemically, for example 6000 IU/day/patient (4).

The object was therefore to seek more effective possibilities of treatment of inflammatory lung diseases and of ARDS, which should make possible a lowering of the pharmaceutical dose previously necessary and thus also an avoidance of systemic side effects. It has now been found that this possibility is opened up by the topical application of antithrombin III by means of inhalation therapy.

The invention therefore relates to a pharmaceutical preparation for inhalation against inflammatory lung diseases and acute pulmonary failure (=ARDS), which contains antithrombin III.

The pharmaceutical preparation according to the invention is either made available in powder form for administration by inhalation or in liquid form for intratracheal or intrabronchial administration. Antithrombin III is present in these pharmaceutical preparations in dissolved or powder form and can be administered as a metered aerosol, as a dry aerosol or as a nasal spray. In addition to antithrombin III, preparations of this type can contain diluents, solvents or alternatively vehicles which facilitate the topical application of antithrombin ll.

Also advantageous are pharmaceutical preparations which contain antithrombin III together with a pulmonary surfactant and/or with an antiinflammatory or a glucocorticoid selected from the group consisting of betamethasone, methylprednisolone and/or dexamethasone. The pulmonary surfactant is preferably a highly purified, natural surfactant made from homogenized porcine lungs or bovine lungs and phospholipids. Liquid pulmonary surfactant preparations are expediently lyophilized before or after the addition of the glucocorticosteroid and then micronized. Compositions according to the invention can contain up to approximately 10% by weight of glucocorticosteroids, up to 40% of pulmonary surfactants and up to 50% of antithrombin ll. It was possible to achieve particularly good therapeutic results using pharmaceutical preparations which contain antithrombin III together with activated protein C, TPFI (=tissue factor pathway inhibitor) or PAF-AH (=platelet-activating factor acylhydrolase). A combination containing other protease inhibitors which inhibit, for example, elastase, such as antitrypsin, SLPI (=secretory leukocyte protease inhibitor), elafin or corresponding synthetic inhibitors is also advantageous.

The preparations according to the invention are administered by inhalation, intratracheally or intrabronchially 3 to 4 times daily for 2 to 4 days.

By means of the pharmaceutical preparation according to the invention, significantly higher active levels of antithrombin can be achieved locally in the lung than by any other administration form. During all previous investigations, it was common that in order to achieve antiinflammatory effects which are most likely mediated by means of an interaction with glycosaminoglycans on the endothelial cell surface, very high systemic concentrations of antithrombin III were necessary in order to confirm the antiinflammatory action (5), but in the case of the topical application according to the invention significantly smaller active compound concentrations suffice.

In clinical investigations, the value of local nebulization of vasodilatory or antiinflammatory substances can be confirmed, where, for example, an improvement in the gas exchange short-term could be achieved by the administration of NO. However, no improvement in the mortality of pulmonary failure could be achieved by the administration of NO by inhalation. On the basis of the assumed mechanisms of action of antithrombin III (local prostacyclin release and down-regulation of antiinflammatory cytokines), it is to be assumed that antithrombin applied locally mediates antiinflammatory effects and possibly even increases the effectiveness of both measures.

### REFERENCES

1. Bernard G. R. et al., The American European Consensus Conference on ARDS: Definitions, mechanisms, relevant outcomes, clinical trial coordination. Am. Ref. Respir. Dis. 1994; 149; 818-24.

2. Pearson U. et al., Surfactant abnormalities in patients with respiratory failure after multiple trauma. Am. Ref. Respir. Dis. 1989; 41: 1033.

3. Tremblay et al., J. Clin. Invest. 1997; 99: 944.

4. Inthorn et al., Shock 1998; 10: 90.

5. Dickneite et al., Semin. Thromb. Hemost 1998; 24: 61.

1. A pharmaceutical preparation for inhalation against inflammatory pulmonary diseases and acute pulmonary failure (=ARDS), which contains antithrombin III.

2. The pharmaceutical preparation as claimed in claim 1, wherein the antithrombin III is present in dissolved or in powder form and can be administered as a metered aerosol, as a dry aerosol or as a nasal spray.

3. The pharmaceutical preparation as claimed in claims 1 and 2, which contains antithrombin III together with diluents, solvents and/or vehicles.

4. The pharmaceutical preparation as claimed in claims 1 to 3, which contains antithrombin III together with an antiinflammatory.

5. The pharmaceutical preparation as claimed in claims 1 to 4, which contains antithrombin III together with activated protein C and/or TFPI (=tissue factor pathway inhibitor) and/or inhibitors of elastase and/or PAF-AH (=platelet-activating factor acylhydrolase).

6. The pharmaceutical preparation as claimed in claim 5, which contains the elastase inhibitor antitrypsin, SLPI (=secretory leukocyte protease inhibitor), elafin or corresponding synthetic inhibitors.

7. The pharmaceutical preparation as claimed in claims 1 to 6, which contains antithrombin III together with pulmonary surfactants and/or with a glucocorticoid selected from the group consisting of betamethasone, methylprednisolone and/or dexamethasone.

8. The use of antithrombin 111 for the production of an inhalant or an aerosol as claimed in claims 1 to 7 for prophylaxis and therapy in acute pulmonary failure.

9. The use of antithrombin 111 for prophylaxis and/or therapy by topical application in inflammatory pulmonary diseases and acute pulmonary failure.
